(19) United States
(12) Patent Application Publication
(10) Pub. No.: US 2024/0335554 A1
(43) Pub. Date: Oct. 10, 2024

(54) POST-TRANSLATIONAL MODIFICATION CHIMERIC MOLECULE

(71) Applicant: UbiquiTx, New York, NY (US)

(72) Inventors: Matthew Barnett, New York, NY (US); Matthew DeLisa, New York, NY (US); Joe Jacobson, New York, NY (US); Pranam Chatterjee, New York, NY (US); Luiz Camargo, New York, NY (US)

(21) Appl. No.: 18/627,132

(22) Filed: Apr. 4, 2024

Related U.S. Application Data
(60) Provisional application No. 63/456,948, filed on Apr. 4, 2023.

(51) Int. Cl. A61K 47/68; A61K 38/45; C12N 9/10; C12N 9/48

(52) U.S. Cl. CPC A61K 47/6815; A61K 38/45; A61K 47/6889; C12N 9/104; C12N 9/485; C12Y 203/02; C12Y 304/19012

(57) ABSTRACT

In one or more aspects, the inventions described herein are directed to chimeric molecules and methods of making the same that provide improved protein modulating processes including, but not limited to, ubiquitination/deubiquination, lipidation/delipidation, SUMOylation/deSUMOylation, nitrosylation/denitrosylation, phosphporylation/dephosphorylation, acetylation/deacetylation, alkylation/dealklyation, methylation/demethylation, carboxylaton/decarboxylation, glycoysylation/deglycoylation, hydroxylation/dehydroxylation, and disulfide bond formation and breakage. In particular, the present disclose provides chimeric molecules including (i) a post-translational modifications (PTMs) domain and (ii) a targeting domain comprising a substrate-binding motif which is heterologous to the PTMS domain, and (iii) a linker that couples the PTMS domain to the targeting domain.

### CROSS REFERENCE TO RELATED APPLICATION

The present application claims the benefit of U.S. patent application Ser. No. 63/456,948, filed Apr. 4, 2023, which is hereby incorporated by reference in its entirety.

### FIELD

The present disclosure relates to targeted post-translation protein modification using chimeras between antibodies and modifying motifs.

### BACKGROUND

To date, therapeutics directed at proteins have generally been restricted to (i) inhibition (by binding a small molecule or large molecule therapeutic to an active site or allosteric modulation site of the target protein) or (ii) degradation (by means of a therapeutic molecule which links the target protein to proteasomal degradation machinery of the cell) limiting the array of potential therapeutic interventions.

However, what is needed in the art is a more generic and holistic approach to targeting protein modification for clinical, investigatory, and therapeutic purposes.

### SUMMARY OF THE INVENTION

In one or more aspects, the present invention expands the therapeutic toolkit by creating a chimeric molecule that can affect a wide array of protein modulating processes including, but not limited to, ubiquitination/deubiquination, lipidation/delipidation, SUMOylation/deSUMOylation, nitrosylation/denitrosylation, phosphporylation/dephosphorylation, acetylation/deacetylation, alkylation/dealklyation, methylation/demethylation, carboxylaton/decarboxylation, glycoysylation/deglycoylation, hydroxylation/dehydroxylation, and disulfide bond formation and breakage.

One aspect of the present disclosure relates to a chimeric molecule comprising (i) a post-translational modifications (PTMs) domain and (ii) a targeting domain comprising a substrate-binding motif which is heterologous to the PTMS domain. A linker couples the PTMS domain to the targeting domain. In some embodiments, the substrate-binding motif and/or the linker has no lysine residues. In some embodiments, the substrate is a protein substrate.

A further aspect of the present disclosure relates to a composition comprising the chimeric molecule and a pharmaceutically-acceptable carrier.

Another aspect of the present disclosure relates to a method of treating a disease. This method involves administering a composition according to the present disclosure to a subject having a disease, where the subject to whom the composition is administered has an increased expression level of a substrate (e.g., a protein substrate) compared to a subject not afflicted with the disease.

Another aspect of the present disclosure relates to a method for protein substrate silencing. This method involves selecting a protein substrate to be silenced and providing a chimeric molecule according to the present disclosure. This method further involves contacting the protein substrate and the chimeric molecule under conditions effective to permit the formation of a protein substrate-molecule complex, where the complex mediates modification or degradation of the protein substrate to be silenced.

Another aspect of the present disclosure relates to forming a ribonucleoprotein. This method involves providing mRNA encoding the chimeric molecule according to the present disclosure and providing one or more polyadenosine binding proteins (“PABP”). This method further involves assembling a ribonucleoprotein complex from the mRNA and the one or more PABPs. In some embodiments, the chimeric molecule is an isolated chimeric molecule.

Another method of the present disclosure relates to a method of screening agents for therapeutic efficacy against a disease. This method involves providing a biomolecule whose presence is mediated by a disease state. A test agent comprising (i) a PTMS domain, (ii) a targeting domain comprising a substrate-binding motif which is heterologous to the PTMS domain, and (iii) a linker coupling the PTMS domain to the targeting domain is also provided. The biomolecule and the test agent are contacted under conditions effective for the test agent to facilitate modification of the biomolecule. The protein structure, function, stability, localization, and interaction of the biomolecule, as a result of the contacting, is determined and the test agent which, based on the determining, decreases the level of the biomolecule is identified as being a candidate for therapeutic efficacy against the disease.

Another aspect of the present disclosure relates to an mRNA molecule encoding a chimeric molecule according to the present disclosure.

A further aspect of the present disclosure relates to a vector encoding an mRNA molecule according to the present disclosure.

A further aspect of the present disclosure relates to an encapsulated nucleic acid molecule comprising: (i) a mRNA molecule according to the present disclosure or a vector according to the present disclosure and (ii) a protein and/or polymer complex.

The present disclosure provides methods and compositions for the creation of engineered chimeras between a synthetic binding protein (e.g., antibodies, DARPins, FN3, monobodies, nanobodies, etc.) and a PTMs domain—that have extended half-life inside of cells.

The present disclosure also provides a chimeric molecule in which the targeting domain is computationally designed.

The present disclosure further provides a chimeric molecule in which the targeting domain is computationally designed and is relatively non-homologous to wild type binders to said target (e.g. a non-natural sequence).

The present disclosure also provides a chimeric molecule in which the PTM domain is computationally designed (e.g. a computationally designed enzyme). For example, and in no way limiting, a chimeric molecule is provided that is designed according to the approaches described in Yeh, A. H W., Norn, C., Kipnis, Y. et al. De novo design of luciferases using deep learning. Nature 614, 774-780 (2023). https://doi.org/10.1038/s41586-023-05696-3 which is herein incorporated by reference as is presented in its entirety.

### BRIEF DESCRIPTION OF THE DRAWINGS

The accompanying drawings, which are incorporated in and constitute a part of this specification, illustrate aspects of the invention and together with the description, serve to explain the principles of the invention.

FIG. 1 is a schematic illustration of a chimeric molecule according to the present invention.

FIG. 2 is a flow diagram detailing the algorithmic creation of one or more specific binders that direct a post translational protein modification to a given biological target.

FIG. 3 is a further flow diagram detailing the algorithmic creation of one or more specific binders that direct a post translational protein modification to a given biological target.

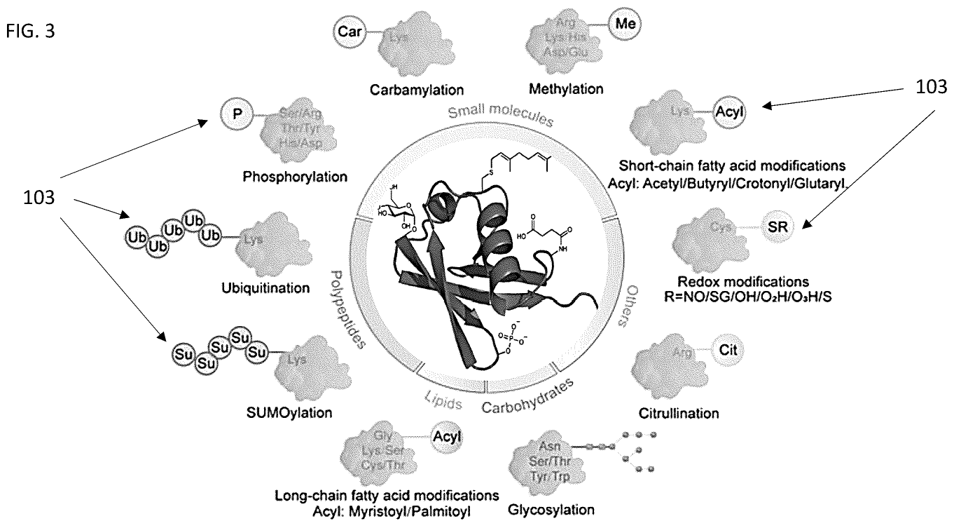

FIG. 4 is a further flow diagram detailing both post translational protein modification targeted protein stabilization of a given biological target.

FIG. 5 is a schematic illustration of a chimeric molecule and a chart detailing the concentration of such molecules.

FIG. 6 details the presence or absence, based on a PTM or protein stabilization of computationally derived chimeric molecules.

FIG. 7 details the selected PTM effect of anti-beta catenin chimeric molecules.

FIG. 8 is graph detailing the off-target binding of anti-beta catenin chimeric molecules.

FIG. 9 is table of biological targets that the computational derived chimeric molecules can be directed against.

### DETAILED DESCRIPTION OF CERTAIN EMBODIMENTS OF THE INVENTION

It is to be appreciated that certain aspects, modes, embodiments, variations, and features of the present disclosure are described below in various levels of detail to provide a substantial understanding of the present technology. The definitions of certain terms as used in this specification are provided below. Unless defined otherwise, all technical and scientific terms used herein generally have the same meaning as commonly understood by one of ordinary skill in the art to which this invention belongs.

In practicing the present invention, many conventional techniques in molecular biology, protein biochemistry, cell biology, immunology, microbiology and recombinant DNA may be used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y. (1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning: the series, Meth. Enzymol., (Academic Press, Inc., 1984); Gene Transfer Vectors for Mammalian Cells, Miller & Calos, Eds. (Cold Spring Harbor Laboratory, New York (1987)); and Meth. Enzymol., Vols. 154 and 155, Wu & Grossman, and Wu, Eds., respectively, which are hereby incorporated by reference in their entirety. Methods to detect and measure levels of polypeptide gene expression products, i.e., gene translation level, are well-known in the art and include the use polypeptide detection methods such as antibody detection and quantification techniques. See also, Strachan & Read, Human Molecular Genetics, Second Edition. (John Wiley and Sons, Inc., New York (1999), all of which are hereby incorporated by reference as if presented in their respective entireties.

As used herein, the term “amino acid” includes naturally-occurring amino acids, L-amino acids, D-amino acids, and synthetic amino acids, as well as amino acid analogs and amino acid mimetics that function in a manner similar to the naturally-occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, e.g., α-carbon that is bound to a hydrogen, a carboxyl group, an amino group, and an R group, e.g., homoserine, norleucine, methionine sulfoxide, methionine methyl sulfonium. Such analogs have modified R-groups, e.g., norleucine, or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally occurring amino acid. Amino acids can be referred to herein by either their commonly known three letter symbols or by the one-letter symbols recommended by the IUPAC-IUB Biochemical Nomenclature Commission.

As used herein the term “antibody” refers to an immunoglobulin and any antigen-binding portion of an immunoglobulin, e.g., IgG, IgD, IgA, IgM and IgE, or a polypeptide that contains an antigen binding site, which specifically or “immunospecifically binds” to, or “immunoreacts with”, an immunogen, antigen, substrate, and the like Antibodies can comprise at least one heavy (H) chain and at least one light (L) chain inter-connected by at least one disulfide bond. The term “V_{H}” refers to a heavy chain variable region of an antibody. The term “V_{L}” refers to a light chain variable region of an antibody. In some embodiments, the term “antibody” specifically covers monoclonal and polyclonal antibodies. A “polyclonal antibody” refers to an antibody which has been derived from the sera of animals immunized with an antigen or antigens. A “monoclonal antibody” refers to an antibody produced by a single clone of hybridoma cells.

Antibody-related molecules, domains, fragments, portions, etc., useful as targeting domains of the invention include, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341:544-546, (1989)), which consists of a V_{H} domain; and (vi) an isolated complementary determining region (CDR). As such “antibody fragments” can comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments. Single-chain antibody molecules may comprise a polymer with a number of individual molecules, for example, dimer, trimer or other polymers.

As used herein, the terms “biomarker” or “biomolecule” or “molecule” refer to a polypeptide (of a particular expression level) which is differentially present in a sample taken from patients having a disease as compared to a comparable sample taken from a control subject or a population of control subjects.

As used herein, the terms “effective amount” or “therapeutically effective amount” of a chimeric molecule or composition is a quantity sufficient to achieve a desired therapeutic and/or prophylactic effect, for example, an amount which results in the prevention of or a decrease in the symptoms associated with a disease that is being treated. The amount of compound administered to the subject will depend on the type and severity of the disease and on the characteristics of the individual, such as general health, age, sex, body weight and tolerance to drugs. It will also depend on the degree, severity or stage of disease. The skilled artisan will be able to determine appropriate dosages depending on these and other factors.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents. Typically, an epitope will be a determinant region form a substrate, which can be recognized by one or more targeting domains.

To screen for targeting domains or substrates which possess an epitope, a routine cross-blocking assay such as that described in Antibodies, A Laboratory Manual, Cold Spring Harbor Laboratory, Ed Harlow and David Lane (1988), which is hereby incorporated by reference in its entirety, can be performed. This assay can be used to determine if a targeting domain binds the same site or epitope of a substrate as a different targeting domain, antibody, antibody fragment and the like. Alternatively, or additionally, epitope mapping can be performed by methods known in the art. For example, the antibody sequence can be mutagenized such as by alanine scanning, to identify contact residues. In a different method, peptides corresponding to different regions of substrate can be used in competition assays with a test target domain or with a test antibody and a target domain or an antibody with a characterized epitope.

As used herein, the term “hypervariable region” refers to the amino acid residues of an antibody which are responsible for antigen-binding. The hypervariable region generally comprises amino acid residues from a “complementarity determining region” or “CDR”, e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest. 5th Ed. Public Health Service, National Institutes of Health, Bethesda, Md. (1991), which is hereby incorporated by reference in its entirety, and/or those residues from a “hypervariable loop” (e.g., residues 26-32 (L1), 50-52 (12) and 91-96 (L3) in the V_{L}, and 26-32 (H1), 52A-55 (H2) and 96-101 (H3) in the V_{H} (Chothia and Lesk J. Mol. Biol. 196:901-917 (1987), which is hereby incorporated by reference in its entirety.

As used herein, the terms “ligand” or “substrate” refer to substances that are able to bind to and form transient or stable complexes with a protein, molecule, chimeric molecule, ligand (dimer), substrate (dimer), a second substrate, a second ligand, target domain, regions, portions, and fragments thereof, ubiquitin or U-box motif regions, domains, or portions thereof, biomolecules, biomarkers, and the like, to serve a biological purpose, for example a substrate which interacts with an enzyme in the process of an enzymatic reaction. Ligands also include signal triggering molecules which bind to sites on a target protein, by intermolecular forces such as ionic bonds, hydrogen bonds and Van der Waals forces. In some embodiments, substrates bind ligands and/or ligands bind substrates.

As used herein, the terms “modification(s)” or “amino acid modification” of a polypeptide, protein, region, domain, or the like, refers to a change in the native sequence such as a deletion, addition, or substation of a desired residue. Such modified polypeptides are prepared by introducing appropriate nucleotide changes into the antibody nucleic acid, or by peptide synthesis. Any combination of deletion, insertion, and substitution is made to obtain the antibody of interest, as long as the obtained antibody possesses the desired properties. The modification also includes the change of the pattern of glycosylation of the protein. A useful method for identification of preferred locations for mutagenesis is called “alanine scanning mutagenesis” as described by Cunningham and Wells in Science, 244:1081-1085 (1989), which is hereby incorporated by reference in its entirety. The mutated antibody is then screened for the desired activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Nevertheless, the monoclonal antibodies to be used in accordance with the present invention may be made by the hybridoma method first described by Kohler et al., Nature 256:495 (1975), which is hereby incorporated by reference in its entirety, or may be made by recombinant DNA methods (see. e.g., U.S. Pat. No. 4,816,567, which is hereby incorporated by reference in its entirety). The “monoclonal antibodies” may also be isolated from phage antibody libraries using the techniques described in Clackson et al., Nature 352:624-628 (1991) and Marks et al., J. Mol. Biol. 222:581-597 (1991), for example, which are hereby incorporated by reference in their entirety.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration.

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

The terms “polypeptide.” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

As used herein, the terms “reference level” or “control level” refer to an amount or concentration of biomarker (or biomolecule, ligand, substrate and the like) which may be of interest for comparative purposes. In some embodiments, a reference level may be the level of at least one biomarker expressed as an average of the level of at least one biomarker taken from a control population of healthy subjects or from a diseased population possessing aberrant expression of a protein or substrate. In another embodiment, the reference level may be the level of at least one biomarker in the same subject at an earlier time, i.e., before the present assay. In even another embodiment, the reference level may be the level of at least one biomarker in the subject prior to receiving a treatment regime.

As used herein, the term “sample” may include, but is not limited to, bodily tissue or a bodily fluid such as blood (or a fraction of blood such as plasma or serum), lymph, mucus, tears, saliva, sputum, urine, semen, stool, CSF, ascities fluid, or whole blood, and including biopsy samples of body tissue. A sample may also include an in vitro culture of microorganisms grown from a sample from a subject. A sample may be obtained from any subject, e.g., a subject/patient having or suspected to have a disease or condition characterized by a disease.

As used herein, the term “screening” means determining whether a chimeric molecule or composition has capabilities or characteristics of preventing or slowing down (lessening) the targeted pathologic condition stated herein, namely a disease or condition characterized by defects in specified disease.

As used herein, the terms “single chain antibodies” or “single chain Fv (scFv)” refer to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). Sec, e.g., Bird et al., Science 242:423-426 (1988) and Huston et al., Proc. Natl. Acad. Sci. USA 85:5879-5883 (1988), which is hereby incorporated by reference in its entirety. Such single chain antibodies are included by reference to the term “antibody” fragments, and can be prepared by recombinant techniques or enzymatic or chemical cleavage of intact antibodies.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with a disease or condition.

As used herein, the term “variable” refers to the fact that certain segments of the variable domains differ extensively in sequence among antibodies. The V domain mediates antigen binding and defines specificity of a particular antibody for its particular antigen. However, the variability is not evenly distributed across the amino acid span of the variable domains. Instead, the V regions consist of relatively invariant stretches called framework regions (FRs) of 15-30 amino acids separated by shorter regions of extreme variability called “hypervariable regions” that are each 9.12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies. See Kabat et al., “Sequences of Proteins of Immunological Interest,” 5th Ed. Public Health Service, National Institutes of Health, Bethesda, Md. (1991), which is hereby incorporated by reference in its entirety). The constant domains are not involved directly in binding an antibody to an antigen, but exhibit various effector functions, such as participation of the antibody in antibody dependent cellular cytotoxicity (ADCC).

As used herein, the terms “variant” or “mutant” are used to refer to a protein or peptide which differs from a naturally occurring protein or peptide, i.e., the “prototype” or “wild-type” protein, by modifications to the naturally occurring protein or peptide, but which maintains the basic protein and side chain structure of the naturally occurring form. Such changes include, but are not limited to: changes in one, few, or even several amino acid side chains; changes in one, few or several amino acids, including deletions, e.g., a truncated version of the protein or peptide, insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A “variant” or “mutant” can have enhanced, decreased, changed, or substantially similar properties as compared to the naturally occurring protein or peptide.

As used herein, the term “ubiquitination” refers to the attachment of the protein ubiquitin to lysine residues of other molecules. Ubiquitination of a molecule, such as a peptide or protein, can act as a signal for its rapid cellular degradation, and for targeting to the proteasome complex.

As used herein, the terms “chimeric molecule” or “ubiquibody” are used interchangeably and refer to a molecule possessing a degradation domain and a targeting domain, attached by a linker region, as defined herein.

As used herein, “deubiquitinating enzymes” or “DUBs” are enzymes that remove ubiquitin molecules from proteins in a process called deubiquitination. Ubiquitin is a small protein that is added to other proteins as a post-translational modification, and this modification can affect protein function, localization, and stability. DUBs play an important role in regulating the ubiquitin system by reversing the effects of ubiquitination. There are many different types of DUBs, each with unique characteristics and functions. Some DUBs remove ubiquitin from single ubiquitinated sites on a protein, while others can cleave entire chains of ubiquitin molecules. DUBs are involved in a wide range of cellular processes, including DNA repair, protein degradation, and immune response. Dysregulation of DUB activity has been linked to a number of diseases, including cancer, neurodegenerative disorders, and inflammatory diseases. In humans there are nearly 100 DUB genes, which can be classified into two main classes: cysteine proteases and metalloproteases. The cysteine proteases comprise ubiquitin-specific proteases (USPs), ubiquitin C-terminal hydrolases (UCHs), Machado-Josephin domain proteases (MJDs) and ovarian tumour proteases (OTU). The metalloprotease group contains only the Jabl/Mov34/Mpr1 Pad1 N-terminal+ (MPN+) (JAMM) domain proteases.

Accordingly, one aspect of the present disclosure relates to a chimeric molecule comprising (i) a degradation domain comprising a deubiquitinating enzymes and (ii) a targeting domain comprising a substrate-binding motif which is heterologous to the deubiquitinating enzyme, and (iii) a linker that couples the PTMs domain to the targeting domain.

An alternative aspect of the present disclosure relates to a chimeric molecule comprising (i) at least onePTMs domain and (ii) a targeting domain comprising a substrate-binding motif which is heterologous to the one or more PTMs domains and (iii) a linker couples the PTMs domain to the targeting domain. In a further arrangement, the at least one PTMs domain includes at least one enzyme directed to ubiquitination, deubiquination, lipidation, delipidation, SUMOylation, deSUMOylation, nitrosylation, denitrosylation, phosphporylation, dephosphorylation, acetylation, deacetylation, alkylation, dealklyation, methylation, demethylation, carboxylaton, decarboxylation, glycoysylation, deglycoylation, hydroxylation, dihydroxylation and disulfide bond formation and breakage.

In some embodiments of the compositions and methods according to the present disclosure, the chimeric molecule (or test agent) is an isolated chimeric molecule (or isolated test agent). As used herein, the terms “isolated” or “purified” polypeptide, peptide, molecule, or chimeric molecule, is substantially free of cellular material or other contaminating polypeptides from the cell or tissue source from which the agent is derived, or substantially free from chemical precursors or other chemicals when chemically synthesized. For example, a chimeric molecule would be free of materials that would interfere with such a molecule's intended function, diagnostic or therapeutic uses. Such interfering materials may include proteins or fragments other than the materials encompassed by the chimeric molecule, enzymes, hormones and other proteinaceous and nonproteinaccous solutes.

In some embodiments of the compositions and methods according to the present disclosure, the linker is heterologous to the PTMs domain and the targeting domain. In accordance with such embodiments, the linker is heterologous to both the PTMs motif of the PTMs domain and the substrate-binding motif of the targeting domain.

As described herein, the substrate-binding motif of the targeting domain is heterologous to a PTMs domain. Accordingly, the PTMs domain may be heterologous to the targeting domain. Likewise, in some embodiments, the PTMs domain does not comprise a substrate-binding motif.

In some embodiments of the compositions and methods according to the present disclosure, the PTMs is a variant of a common or classically known PTMs. As described herein supra, the term “variant” refers to a protein or peptide which differs from a naturally occurring protein or peptide, i.e., the “prototype” or “wild-type” protein, by modifications to the naturally occurring protein or peptide, but which maintains the basic protein and side chain structure of the naturally occurring form.

The terms “degradation domain” or “degradation region” are used interchangeably and refer to a portion of a chimeric molecule that can facilitate the ubiquitination or deubiquitination of a substrate. As described herein, the degradation domain includes, in one or more particular implementations, an E3 ubiquitin ligase motif without lysine residues.

The terms “targeting domain” or “target domain” or “targeting moiety” are used interchangeably and refer to a polypeptide region bound covalently or non-covalently to another region within a chimeric molecule, which may enhance the concentration of the chimeric molecule or composition in a target sub-cellular location, cell, or tissue relative, as compared to the surrounding locations, cells, and/or tissue.

As used herein, the term “PTMs”, or “post-translational modification(s)” refers to chemical modifications that occur on a protein after it has been synthesized. These modifications can affect protein structure, function, stability, localization, and interactions with other molecules.

In accordance with one or more implementations of the present invention, the therapeutic toolkit afforded by using such PTMs can include degradation using E3 ligases, such as those provided in U.S. Pat. No. 11,192,942, which is incorporated herein by reference as is presented in its entirety. However, the chimeric molecule of the present disclosure can be configured to provide a wide array of protein modulating processes including, but not limited to, ubiquitination/deubiquination, lipidation/delipidation, SUMOylation/deSUMOylation, nitrosylation/denitrosylation, phosphporylation/dephosphorylation, acetylation/deacetylation, alkylation/dealklyation, methylation/demethylation, carboxylaton/decarboxylation, glycoysylation/deglycoylation, hydroxylation/dehydroxylation, and disulfide bond formation and breakage.

As described, the chimeric molecules of the present disclosure include a PTMs domain. Here, the PTMs domain can refer compounds or compositions that are directed to carry out: Phosphorylation or other additions of a phosphate group to serine, threonine, or tyrosine residues by kinases; glycosylation or other additions of carbohydrate groups (glycans) to asparagine, serine, or threonine residues by glycosyltransferases. The PTMs domain can refer compounds or compositions that are directed to carry out acetylation or other additions of an acetyl group to the amino terminus of a protein or to lysine residues by acetyltransferases; methylation or other additions of a methyl group to lysine or arginine residues by methyltransferases. As noted, The PTMs domain can refer compounds or compositions that are directed to carry out ubiquitination or addition of one or more ubiquitin molecules to lysine residues by ubiquitin ligases; sumoylation or addition of one or more small ubiquitin-like modifier (SUMO) molecules to lysine residues by SUMO ligases; palmitoylation or other additions of a fatty acid (palmitate) to cysteine residues by palmitoyltransferases. F

In further arrangements, the PTMs domain can refer compounds or compositions that are directed to carry out proteolytic cleavage or other cleavages of proteins by proteases to generate smaller, active fragments; ADP-ribosylation or other additions of an ADP-ribose group to proteins by ADP-ribosyltransferases, phosphopantetheinylation or other attachments of a 4′-phosphopantetheine group to a serine residue by phosphopantetheinyl transferases.

It is appreciated and understood that the PTMs domain can include additional compositions. For example, the PTMs domain can refer compounds or compositions that are directed to carry out myristoylation or other attachments of myristate, palmitoylation; isoprenylation or prenylation, the addition of an isoprenoid group (e.g. farnesol and geranylgeraniol); farnesylation; geranylgeranylation; glypiation, glycosylphosphatidylinositol (GPI); lipoylation; phosphopantetheinylation; diphthamide formation (on a histidine found in cEF2); ethanolamine phosphoglycerol attachment; hypusine formation; beta-Lysine addition on a conserved lysine of the elongation factor P (EFP) in most bacteria. The PTMs domain can refer compounds or compositions that are directed to carry out acylation, N-acylation (amides), S-acylation (thioesters); or acetylation.

Further PTMs can include deacetylation; formylation; alkylation, demethylation; and arginylation. Furthermore, PTMs include tRNA-mediation addition such as polyglutamylation, polyglycylation, butyrylation; and glycosylation.

Additional PTMs can include compositions directed to polysialylation; malonylation; hydroxylation; iodination; phosphorylation; adenylylation; uridylylation; propionylation; pyroglutamate formation; S-glutathionylation; S-nitrosylation; S-sulfenylation; S-sulfinylation; S-sulfonylation; succinylation; or sulfation.

Other PTMs can refer to compounds or compositions that are directed to carry out glycation and carbamylation, biotinylation; carbamylation; oxidation; pegylation; SUMOylation; neddylation; ISGylation; pupylation; citrullination, deimination, deamidation; and eliminylation.

Furthermore, more PTMs can include compositions that are directed to carry out structural changes; disulfide bridges; proteolytic cleavage; isoaspartate formation; racemization; protein splicing, or as otherwise provided in the following list of references https://en.wikipedia.org/wiki/Post-translational_modification].

The targeting domains of the present disclosure may be monospecific, bispecific, trispecific, or of greater multispecificity. Multispecific targeting domains can be specific for different epitopes of a substrate or can be specific for both a substrate polypeptide of the present invention as well as for heterologous compositions, such as a heterologous polypeptide or solid support material. See, e.g., WO 93/17715; WO 92/08802; WO 91/00360; WO 92/05793; Tutt et al., “Trispecific F(ab′)3 Derivatives that use Cooperative Signaling via the TCR/CD3 Complex and CD2 to Activate and Redirect Resting Cytotoxic T Cells,” J. Immunol. 147:60-69 (1991); U.S. Pat. Nos. 5,573,920, 4,474,893, 5,601,819, 4,714,681, 4,925,648; 6,106,835; Kostelny et al., “Formation of a Bispecific Antibody by the Use of Leucine Zippers,” J. Immunol. 148:1547-1553 (1992), all of which are herein incorporated by reference in their respective entireties.

The targeting domains of the present disclosure can be from any animal origin, including birds and mammals. For example, the targeting domains may be from human, marine, rabbit, goat, guinea pig, camel, horse, or chicken.

Techniques for generating targeting domains directed to target substrates are well known to those skilled in the art. Examples of such techniques include, but are not limited to, e.g., those involving display libraries, xeno or humab mice, hybridomas, and the like. Target polypeptides—from which a targeting domain is derived—within the scope of the present disclosure include any polypeptide or polypeptide derivative which is capable of exhibiting antigenicity. Examples include, but are not limited to, substrate and fragments thereof.

In some embodiments of the compositions and methods according to the present disclosure, the targeting domain is derived from or is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d.

In some embodiments of the compositions and methods according to the present disclosure, the targeting domain is a single chain antibody. Single chain antibodies (“scFv”) are genetically engineered antibodies that consist of the variable domain of a heavy chain at the amino terminus joined to the variable domain of a light chain by a flexible region. In some embodiments, scFv are generated by PCR from hybridoma cell lines that express monoclonal antibodies (mAbs) with known target specificity, or they are selected by phage display from libraries isolated from spleen cells or lymphocytes, and preserve the affinity of the parent antibody. Employing a protocol to identify intracellular substrates, the yeast two-hybrid technology serves to identify candidate scFv-protein interactions. Such a system is useful to predict whether or not a scFv will be able to recognize its target substrate in vivo (see Pörtner-Taliana et al., “Identification of Protein Single chain Antibody Interactions In Vivo Using Two-hybrid Protocols,” Protein-Protein Interactions: A Molecular Cloning Manual, Cold Spring Harbor Laboratory Press, Chapter 24 (2002), which is hereby incorporated by reference in its entirety.

Typically, scFv, hybrid antibodies or hybrid antibody fragments that are cloned into a display vector can be selected against the appropriate antigen to identify variants that maintained good binding activity, because the antibody or antibody fragment will be present on the surface of the phage or phagemid particle (see, e.g., Barbas III et al., Phage Display, A Laboratory Manual (Cold Spring Harbor Laboratory Press, Cold Spring Harbor, N.Y., 2001), which is hereby incorporated by reference in its entirety). However, other vector formats could be used for this process, such as cloning the antibody fragment library into a lytic phage vector (modified T7 or Lambda Zap systems) for selection and/or screening.

In some embodiments of the compositions and methods according to the present disclosure, where the targeting domain is a monobody, the monobody may be a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), EI3.4.3 (EGFR), EI4.2.1 (EGFR), EI4.4.2 (EGFR), EI6.2.6 (EGFR), EI6.2.10 (EGFR), E246 (EGFR), C743 (CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), ml2.2.1 (mIgG), HA4 (AbISH2), HA10 (AbISH2), HA16 (AbISH2), HA18 (AblSH2), 159 (vEGFR), MUC16 (MSLN), E2 #3 (ERα/EF), E2 #4 (ERα/EF), E2 #5 (ERα/EF), E2 #6 (ERα/EF), E2 #7 (ERα/EF), E2 #8 (ERα/EF), E2 #9 (ERα/EF), E2 #10 (ERα/EF), E2 #11 (ERα/EF), E2 #23 (ERα/EF), E3 #2 (ERα/EF), E3 #6 (ERα/EF), OHT #31 (ERα/EF), OHT #32 (ERα/EF), OHT #33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avB3 integrin), 1C9 (Src SH3), IF11 (Src SH3), IF10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozymc), BF4.9 (Lysozymc), BF4.4 (Lysozyme), BFslc4.01 (Lysozyme), BFslc4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

The targeting domain according to the present disclosure comprises a substrate-binding motif which is heterologous to the PTMs domain. For example, where the degradation domain includes E3 ubiquitin ligase the substrate binding motif is heterologous to the degradation domain. The substrate-binding motif may recognize a protein substrate (e.g., a biomolecule). In accordance with such embodiments the E3 ubiquitin ligase motif of the degradation domain permits ubiquination of the protein substrate (e.g., the biomolecule).

As described herein, a known or unknown substrate (e.g., a protein substrate) may be bound by the substrate-binding motif of the targeting domain for subsequent post translational modification. The substrates include, but are not limited to, intracellular substrates, extracellular substrates, modified substrates, glycosylated substrates, farnesylated substrates, post translationally modified substrates, phosphorylated substrates, and other modifications known in the art. In some embodiments, the substrate is an intracellular substrate, e.g., an intracellular protein substrate.

In some embodiments of the compositions and methods according to the present disclosure, the substrates include, but are not limited to, β-galactosidase, fluorescent protein, histone protein, nuclear localization signal (NLS), H-Ras protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

In some embodiments of the compositions and methods according to the present disclosure, the substrate is a fluorescent protein selected from the group consisting of green fluorescent protein, emerald fluorescent protein, venus fluorescent protein, cerulean fluorescent protein, and enhanced cyan fluorescent protein.

Although targeting domains possess intrinsic binding interactions, e.g., secondary, tertiary, or quaternary flexibility, there must still be flexibility with respect to the association with the PTMs domain. For example, where the PTMs domain is a degradation domain using E3 ubiquitin, absence adequate spacing, it is possible for the E3 ubiquitin ligase motif of such a degradation domain to sterically hinder the substrate-targeting domain interaction. As such, the present disclosure employs polypeptide linkers of sufficient length to prevent the steric disruption of binding between the targeting domain and the substrate, in some embodiments.

In some embodiments of the compositions and methods according to the present disclosure, the targeting domain is covalently attached to the PTMs domain via a linker that may be cleavable or non-cleavable under physiological conditions. The linker can entail an organic moiety comprising a nucleophilic or electrophilic reacting group which allows covalent attachment of the degradation domain to the targeting domain. In some embodiments, the linker is an enol ether, ketal, imine, oxime, hydrazone, semicarbazone, acylimide, or methylene radical. The linker may be an acid-cleavable linker, a hydrolytically cleavable linker, or enzymatically-cleavable linker, in some embodiments.

Accordingly, in some embodiments of the compositions and methods according to the present disclosure, when the linker is cleavable, the linker may be enzymatically or hydrolytically cleavable.

Peptide-based linking groups are cleaved by enzymes such as peptidases and proteases in cells. Peptide-based cleavable linking groups are peptide bonds formed between amino acids to yield oligopeptides, e.g., dipeptides, tripeptides, and polypeptides. Peptide-based cleavable groups do not include the amide group (—C(O) NH—). The amide group can be formed between any alkylene, alkenylene, or alkynelene. A peptide bond is a special type of amide bond formed between amino acids to yield peptides and proteins. The peptide-based cleavage group is generally limited to the peptide bond, i.e., the amide bond, formed between amino acids yielding peptides and proteins and does not include the entire amide functional group. Peptide cleavable linking groups have the general formula —NHCHR1C(O)NHCHR2C(O)—, where R1 and R2 are the R groups of the two adjacent amino acids. These candidates can be evaluated using methods analogous to those described above.

For in vitro applications, appropriate linkers, which can be cross-linking agents for use for conjugating a polypeptide to a solid support, include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl) propionic acid can be employed as a means for cleaving a polypeptide from a solid support. See Brown et al., Mol. Divers 4-12 (1995); Rothschild et al., Nucl. Acids Res. 24:351-66 (1996); and U.S. Pat. No. 5,643,722), which are hereby incorporated by reference in their respective entireties.

In some embodiments of the compositions and methods according to the present disclosure, the linker is not cleavable.

In some embodiments of the compositions and methods according to the present disclosure, the linker is heterologous to the PTMs domain and the targeting domain.

An antibody, polypeptide, or fragment thereof, such as a targeting domain, can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoracetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

It will be readily apparent to the skilled artisan that the methods and techniques described above can be employed for the chimeric molecule of the present invention, including its constituent parts, and modifications thereof, as well as the linker molecules, as described above.

A further aspect of the present disclosure relates to a composition comprising the chimeric molecule and a pharmaceutically-acceptable carrier. Such compositions generally entail recombinant or substantially purified chimeric molecules and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the protein compositions (see, e.g., Remington's Pharmaceutical Sciences, Mack Publishing Co., Easton, Pa. 18th ed. (1990), which is hereby incorporated by reference in its entirety). The pharmaceutical compositions are generally formulated as sterile, substantially isotonic and in full compliance with all Good Manufacturing Practice (GMP) regulations of the U.S. Food and Drug Administration.

The compositions of the present disclosure can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The compositions of the present disclosure may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compositions may be incorporated with excipients and used in the form of tablets, capsules, elixirs, suspensions, syrups, and the like. Such compositions and preparations should contain at least 0.1% of active compound. The percentage of the composition in these compositions may, of course, be varied and may conveniently be between about 2% to about 60% of the weight of the unit. Preferred compositions according to the present invention are prepared so that an oral dosage unit contains between about 1 and 250 mg of the chimeric molecule according to the present disclosure.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

These compositions may also be administered parenterally. Solutions or suspensions of the present compositions can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The composition of the present disclosure may also be administered directly to the airways in the form of an aerosol. For use as aerosols, the compositions of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compositions of the present disclosure may further contain, in some embodiments, a second agent or pharmaceutical composition selected from the non-limiting group of anti-inflammatory agents, antidiabetic agents, hpyolipidemic agents, chemotherapeutic agents, antiviral agents, antibiotics, metabolic agents, small molecule inhibitors, protein kinase inhibitors, adjuvants, apoptotic agents, proliferative agents, organotropic targeting agents, immunological agents, antigens from pathogens, such as viruses, bacteria, fungi and parasites, optionally in the form of whole inactivated organisms, peptides, proteins, glycoproteins, carbohydrates, or combinations thereof, any examples of pharmacological or immunological agents that fall within the above-mentioned categories and that have been approved for human use that may be found in the published literature, any other bioactive component, or any combination of any of these.

In some embodiments, the composition further contains a second agent selected from the group consisting of an anti-inflammatory agent, an antidiabetic agent, a hypolipidemic agent, a chemotherapeutic agent, an antiviral agent, an antibiotic, a metabolic agent, a small molecule inhibitor, a protein kinase inhibitor, adjuvants, apoptotic agents, a proliferative agent, and organotropic targeting agents, and any combination thereof.

Another aspect of the present disclosure relates to a method of treating a disease. This method involves administering a composition according to the present disclosure to a subject having a disease, where the subject to whom the composition is administered has an increased expression level of a substrate (e.g., a biomolecue) compared to a subject not afflicted with the disease.

The treatment methods of the present disclosure may involve administering a composition according to the present disclosure to a subject, where the disease possesses a measurable phenotype. In some embodiments, the phenotype of the disease involves an increased expression level of a substrate compared to the phenotype from a subject not afflicted with the disease. In this respect, chimeric molecules contained in the pharmaceutical compositions of the present disclosure are efficacious against treating or alleviating the symptoms from a disease characterized by a phenotypic increase in the expression level of one or more substrates compared to the phenotype from a subject not afflicted with the disease.

Non-limiting examples of diseases that can be treated or prevented in the context of the present invention, include, cancer, metastatic cancer, solid cancers, invasive cancers, disseminated cancers, breast cancer, lung cancer, NSCLC cancer, liver cancer, prostate cancer, brain cancer, pancreatic cancer, lymphatic cancer, ovarian cancer, endometrial cancer, cervical cancer, and other solid cancers known in the art, blood cell malignancies, lymphomas, leukemias, myelomas, stroke, ischemia, myocardial infarction, congestive heart failure, stroke, ischemia, peripheral vascular disease, alcoholic liver disease, cirrhosis, Parkinson's disease, Alzheimer's disease, diabetes, cancer, arthritis, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, septic shock, multiple organ dysfunction syndrome, rheumatoid arthritis, trauma, stroke, heart infarction, systemic autoimmune disease, chronic hepatitis, overweight, and/or obesity, or any combination thereof.

In some embodiments, the disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

When used in vivo for therapy, the compositions are administered to the subject in effective amounts, i.e., amounts that have desired therapeutic effect. The dose and dosage regimen will depend upon the degree of the disease in the subject, the characteristics of the particular chimeric molecule used, e.g., its therapeutic index, the subject, and the subject's history. The effective amount may be determined during pre-clinical trials and clinical trials by methods familiar to physicians and clinicians. An effective amount of a peptide useful in the methods may be administered to a mammal in need thereof by any of a number of well-known methods for administering pharmaceutical compounds.

Dosage, toxicity and therapeutic efficacy of the compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices may be desirable. While compositions that exhibit toxic side effects may be used, care should be taken to design a delivery system that targets such compositions to the site of affected tissue in order to minimize potential damage to uninfected cells and, thereby, reduce side effects.

In some embodiments, administering the compositions of the present disclosure is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

In some embodiments, suitable in vitro or in vivo assays are performed to determine the effect of the chimeric molecules and compositions of the present disclosure and whether administration is indicated for treatment. Compositions for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

Any method known to those in the art for contacting a cell, organ or tissue with a composition may be employed. In vivo methods typically include the administration of a chimeric molecule or composition, such as those described above, to a mammal, suitably a human. When used in vivo for therapy, the chimeric molecules or compositions are administered to the subject in effective amounts, as described herein. Results can be ascertained as per the empirical variables set forth at the outset of the methods described herein.

In vitro methods typically include the assaying the effect of chimeric molecule or composition, such as those described above, on a sample or extract. In some embodiments, chimeric molecule efficacy can be determined by assessing the effect on substrate degradation, i.e., the ability of the chimeric molecules and compositions to exert a phenotypic change in a sample. Such methods include, but are not limited to, immunohistochemistry, immunofluorescence, ELISPOT, ELISA, or RIA. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods.

Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, immunohistochemistry, fluorescence microscopy, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomolecule) with an antibody to the molecule of interest or contacting an antibody to a molecule of interest (such as antibodies to the disclosed biomolecule) with a molecule that can be bound by the antibody, as the case may be, under conditions effective to allow the formation of immunocomplexes. In this regard, the skilled artisan will be able to assess the presence and or level of specific biomolecules in a given sample. Subsequently, the chimeric molecule compositions of the present invention are added to the assay. Thereafter, the level of biomolecule can be assessed, i.e., the presence or level thereof, using the immunoassays described herein to determine the post-treatment phenotypic effect.

Immunoassays can include methods for detecting or quantifying the amount of a biomolecule of interest in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label (see, e.g., U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

Another aspect of the present disclosure relates to a method for protein substrate silencing. This method involves selecting a protein substrate to be silenced and providing a chimeric molecule according to the present disclosure. This method further involves contacting the protein substrate and the chimeric molecule under conditions effective to permit the formation of a protein substrate-molecule complex, where the complex mediates degradation of the protein substrate to be silenced. In some embodiments, the complex mediates the degradation by post-translational ubiquitination of the substrate.

The methods involve silencing one or more substrates previous described herein. In some embodiments, the protein substrate is selected from the group consisting of β-galactosidase, fluorescent protein, histone protein, nuclear localization signal (NLS), H-Ras protein, SHP2 protein, Src-homology 2 domain-containing phosphatase 2 (SHP2), β-galactosidasc, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

In some embodiments, the substrate is a fluorescent protein selected from the group consisting of green fluorescent protein, emerald fluorescent protein, venus fluorescent protein, cerulean fluorescent protein, and enhanced cyan fluorescent protein.

Another aspect of the present disclosure relates to forming a ribonucleoprotein. This method involves providing a mRNA encoding the chimeric molecule according to the present disclosure and providing one or more polyadenosine binding proteins (“PABP”). This method further involves assembling a ribonucleoprotein complex from the mRNA and the one or more PABPs. In some embodiments, the mRNA comprises a 3′-terminal polyadenosine (poly A) tail.

Another method of the present disclosure relates to a method of screening agents for therapeutic efficacy against a disease. This method involves providing a biomolecule whose presence is mediated by a disease state. A test agent comprising (i) a PTMs domain without lysine residues, (ii) a targeting domain comprising a substrate-binding motif which is heterologous to the PTMs domain, and (iii) a linker coupling the PTMs domain to the targeting domain are provided. The biomolecule and the test agent are contacted under conditions effective for the test agent to facilitate degradation of the biomolecule. The level of the biomolecule, as a result of the contacting, is determined and the test agent which, based on the determining, decreases the level of the biomolecule is identified as being a candidate for therapeutic efficacy against the disease. Accordingly, in some embodiments, the identified test agent comprises a substrate-binding motif which binds the biomolecule.

There are a myriad of diseases in which the degree of overabundance of certain biomolecules are known to be indicative of whether a subject is afflicted with a disease or is likely to develop a disease (see, e.g., Anderson et al., “Discovering Robust Protein Biomarkers for Disease from Relative Expression Reversals in 2-D DIGE Data,” Proteomics 7:1197-1207 (2007), which is hereby incorporated by reference in its entirety). Accordingly, in some embodiments, the biomolecule is associated with cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, or obesity, and any combination thereof.

In some embodiments, the test agent is a chimeric molecule according to the present disclosure.

As described herein, the test agent comprises a PTMs domain coupled to a targeting domain by a linker. In some embodiments, the linker is a polypeptide linker of sufficient length to prevent the steric disruption of binding between said targeting domain and said biomolecule. The linker may be heterologous to the PTMs domain and the targeting domain.

In some embodiments, the PTMs domain, the targeting domain, and/or the linker coupling the PTMs domain to the targeting domain have no lysine residues. In some embodiments, the test agent comprises no lysine residues.

Identifying the test agent may be carried out with respect to a standard biomolecule level in a subject not afflicted with said disease.

In some embodiments, identifying the test agent may be carried out with a plurality of test agents.

Targeting domains and substrate-binding motifs for use in methods of the present disclosure are described in detail supra. In some embodiments, the targeting domain and/or the substrate-binding motif is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d. Accordingly, the targeting domain and/or the substrate-binding motif may be a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), EI3.4.3 (EGFR), EI4.2.1 (EGFR), EI4.4.2 (EGFR), EI6.2.6 (EGFR), EI6.2.10 (EGFR), E246 (EGFR), C743 (CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), ml2.2.1 (mIgG), HA4 (AbISH2), HA10 (AbISH2), HA16 (AbISH2), HA18 (AblSH2), 159 (vEGFR), MUC16 (MSLN), E2 #3 (ERα/EF), E2 #4 (ERα/EF), E2 #5 (ERα/EF), E2 #6 (ERα/EF), E2 #7 (ERα/EF), E2 #8 (ERα/EF), E2 #9 (ERα/EF), E2 #10 (ERα/EF), E2 #11 (ERα/EF), E2 #23 (ERα/EF), E3 #2 (ERα/EF), E3 #6 (ERα/EF), OHT #31 (ERα/EF), OHT #32 (ERα/EF), OHT #33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avB3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozymc), 2L4.1 (Lysozyme), BF4.1 (Lysozyme), BF4.9 (Lysozyme), BF4.4 (Lysozyme), BFslc4.01 (Lysozyme), BFslc4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), g12.5.3T88I (goat IgG), g12.5.2 (goat IgG), g12.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

In accordance with this aspect of the disclosure, the substrate may be selected from the group consisting of β-galactosidase, fluorescent protein, histone protein, nuclear localization signal (NLS), H-Ras protein, SHP2 protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

In some embodiments, the substrate is a fluorescent protein selected from the group consisting of green fluorescent protein, emerald fluorescent protein, venus fluorescent protein, cerulean fluorescent protein, and enhanced cyan fluorescent protein.

In some embodiments, the substrate is a fusion protein comprising a fluorescent protein. Suitable fluorescent proteins are identified supra.

Another aspect of the present disclosure relates to a method of screening for disease biomarkers. This method involves providing a sample of diseased cells expressing one or more ligands. A plurality of chimeric molecules comprising (i) a PTMs domain, (ii) a targeting domain comprising a substrate-binding motif which is heterologous to the PTMs, and (iii) a linker coupling the PTMs domain to the targeting domain are provided. This method further involves contacting the sample with the plurality of chimeric molecules under conditions effective for the diseased cells to fail to proliferate in the absence of the chimeric molecule, determining which of the chimeric molecules permit the diseased cells to proliferate, and identifying, as biomarkers for the disease, based on the determining the ligands which bind to the chimeric molecules and permit diseased cells to proliferate.

Many, if not all diseases, are complex and multifactorial. When considering neurodegeneration, for example, substantial neuronal cell loss occurs before pathologic presentation. Screening for and developing such drugs—to treat neurodegenerative diseases—is further stymied by ancillary therapies which ameliorate the symptoms. Thus, target detection is obfuscated by prior therapeutic administration, which, may in turn, slow disease progression and further confound treatment regimes. In this way, the present disclosure provides new, inventive, screening methods for elucidation of disease biomarkers by employing phenotypic screening analyses (see, e.g., Pruss, R. M., “Phenotypic Screening Strategies for Neurodegenerative Diseases: A Pathway to Discover Novel Drug Candidates and Potential Disease Targets or Mechanisms,” CNS &Neurological Disorders—Drug Targets, 9, 693-700 (2010), which is hereby incorporated by reference in its entirety).

Phenotypic screening involves using an appropriate sample, e.g., class of cells, cell extract, neurons, tissue, and the like, from a patient afflicted with a disease and subjecting the sample to one or more chimeric molecules as described herein. Subsequently, the sample is screened for viability, proliferation, cell processes and/or phenotypic characteristic of the diseased cell, e.g., shrinking, loss of membrane potential, morphological changes, and the like. See id. Image analysis software allows for cell bodies or other objects to empirically assess the results. Hits coming from the screen may maintain cell survival by stimulating survival pathways, mimicking trophic factors, or inhibiting death signaling. Higher content screening and profiling in target-directed secondary assays can then be used to identify targets and mechanisms of action of promising hits.

Examples of diseases conditions from which a biomarker screening analysis can be performed include the diseases described above. In some embodiments, the disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

In some embodiments, the method of screening for disease biomarkers includes a plurality of chimeric molecules, where the molecules possess an E3 ubiquitin ligase motif, as described supra. In some embodiments, the biomarker screening method includes a plurality of chimeric molecules possessing a targeting domain, as described above. The screening methods of the present disclosure employ polypeptide linkers of sufficient length to prevent the steric disruption of binding between the targeting domain and the ligand.

Once a chimeric molecule is determined to provide a therapeutic indication, the biomarker is isolated using the targeting domain region (or the entire chimeric molecule) to immunoprecipitate the biomarker, from a sample, which is subsequently identified using methods well known in the art. Biomarker isolation and purification methods include, but are not limited to, for example, HPLC or FPLC chromatography using size-exclusion or affinity-based column resins (see, e.g., Sambrook, et al. 1989, Cold Spring Harbor Laboratory Press, which is hereby incorporated by reference in its entirety).

Active fragments, derivatives, or variants of the polypeptides of the present disclosure may be recognized by, for example, the deletion or addition of amino acids that have minimal influence on the properties, secondary structure, and biological activity of the polypeptide. For example, a polypeptide may be joined to a signal (or leader) sequence at the N-terminal end of the protein which co-translationally or post-translationally directs sub-cellular or extracellular localization of the protein.

The biomarker can then be elucidated using techniques known in the art. In some embodiments, determining the identity of the biomarker is performed using MALDI-TOF, mass spectrometry, mass spectroscopy, protein sequencing, antibody interactions, western blot, immunoassay, ELISA, chromatographic techniques, reverse proteomics, immunoprecipitations, radioimmunoassay, and immunofluorescence, or any combinations thereof.

Suitable mass spectrometric techniques for the study and identification of proteins include, laser desorption ionization mass spectrometry and electrospray ionization mass spectrometry. Within the category of laser desorption ionization (LDI) mass spectrometry (MS), both matrix assisted LDI (MALDI) and surface assisted LDI (SELDI) time-of-flight (TOF) MS may be employed. SELDI TOF-MS is particularly well-suited for use in the present methods because it provides attomole sensitivity for analysis, quantification of low abundant proteins (pg-ng/ml) and highly reproducible results.

The methods described herein can be performed, e.g., by utilizing pre-packaged kits comprising at least one reagent, e.g., a chimeric molecule or composition described herein, which can be conveniently used, e.g., in clinical settings to treat subjects exhibiting symptoms of a disease or illness involving an overexpressed substrate, biomolecule, or biomarker.

Another aspect of the present disclosure relates to a mRNA molecule encoding a chimeric molecule according to the present disclosure. Suitable chimeric molecules are described in detail supra. The mRNA molecule may comprise a 3′-terminal polyadenosine (poly A) tail.

A further aspect of the present disclosure relates to a vector encoding a mRNA molecule according to the present disclosure.

The vector may be an expression vector. In general, expression vectors useful in recombinant DNA techniques are often in the form of plasmids. However, the present disclosure is intended to include such other forms of expression vectors that are not technically plasmids, such as viral vectors, e.g., replication defective retroviruses, adenoviruses and adeno-associated viruses, which serve equivalent functions. Such viral vectors permit infection of a host cell and expression in that host cell of, e.g., a mRNA and its subsequent translation to a protein.

In some embodiments, the vector is a viral vector, e.g., an adenoviral vector, an adeno-associated virus vector, or a lentiviral vector.

The vectors may comprise eukaryotic promoter systems capable of transforming or transfecting eukaryotic host cells. Once the vector has been incorporated into the appropriate host, the host is maintained under conditions suitable for high level expression of the nucleotide sequences encoding the chimeric molecule according to the present disclosure. Vectors can also encode a signal peptide, e.g., pectate lyase, useful to direct the secretion of extracellular antibody fragments (see U.S. Pat. No. 5,576,195, which is hereby incorporated by reference in its entirety).

Expression of the chimeric molecules of the present disclosure in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression; (ii) to increase the solubility; and (iii) to aid in purification by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their endogenous recognition sequences, include Factor Xa, thrombin and enterokinase. Typical fusion expression vectors include pGEX (Pharmacia Biotech Inc; Smith and Johnson, Gene 67:31-40 (1988), which is hereby incorporated by reference in its entirety), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., “Tightly Regulated tac Promoter Vectors useful for the Expression of Unfused and Fused Proteins in Escherichia coli,” Gene 69 (2): 301-315 (1988), which is hereby incorporated by reference in its entirety) and pET 11d (Studier et al., Gene Expression Technology: Methods In Enzymology 185, Academic Press, San Diego, Calif. 60-89 (1990), which is hereby incorporated by reference in its entirety). Methods for targeted assembly of distinct active peptide or protein domains to yield multifunctional polypeptides via polypeptide fusion has been described by U.S. Pat. Nos. 6,294,353 and 6,692,935, which are hereby incorporated by reference in their entirety. One strategy to maximize recombinant polypeptide expression, e.g., a chimeric molecule of the present disclosure, in E. coli is to express the polypeptide in host bacteria with an impaired capacity to proteolytically cleave the recombinant chimera (see, e.g., Gottesman, Gene Expression Technology: Methods In Enzymology 185, Academic Press, San Diego, Calif. 119-128 (1990), which is hereby incorporated by reference in its entirety).

Expression of the chimeric molecules of the present disclosure may be carried out in mammalian cells using a mammalian expression vector. Examples of mammalian expression vectors include, e.g., but are not limited to, pCDM8 (Seed, “An LFA-3 cDNA Encodes a Phospholipid-Linked Membrane Protein Homologous to its Receptor CD2,” Nature 329:840 (1987), which is hereby incorporated by reference in its entirety) and pMT2PC (Kaufman, et al., “Translational Efficiency of Polycistronic mRNAs and their Utilization to Express Heterologous Genes in Mammalian Cells,” EMBO J. 6:187-195 (1987), which is hereby incorporated by reference in its entirety). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells useful for expression of the chimeric molecules according to the present disclosure (see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., (1989), which are hereby incorporated by reference in their entirety).

A further aspect of the present disclosure relates to an encapsulated nucleic acid molecule comprising: (i) a mRNA molecule according to the present disclosure or a vector according to the present disclosure and (ii) a protein and/or polymer complex.

In some embodiments, the nucleic acid is a mRNA molecule, wherein said mRNA molecule comprises a 3′-terminal polyadenosine (poly A) tail, and wherein said protein complex comprises one or more polyadenosine binding proteins (“PABP”).

In some embodiments, the protein and/or polymer complex comprises one or more antibodies, antibody derivatives, antibody-drug conjugates, phage proteins, ubiquibodies, or combinations thereof.

In some embodiments, the protein and/or polymer complex forms a nanoplex.

As noted, therapeutics directed at proteins have generally been restricted to (i) inhibition (by binding a small molecule or large molecule therapeutic to an active site or allosteric modulation site of the target protein) or (ii) degradation (by means of a therapeutic molecule which links the target protein to proteasomal degradation machinery of the cell) limiting the array of potential therapeutic interventions. The present invention expands the therapeutic toolkit by creating a chimeric molecule that can affect a wide array of protein modulating processes including, but not limited to, ubiquitination/deubiquination, lipidation/delipidation, SUMOylation/deSUMOylation, nitrosylation/denitrosylation, phosphporylation/dephosphorylation, acetylation/deacetylation, alkylation/dealklyation, methylation/demethylation, carboxylaton/decarboxylation, glycoysylation/deglycoylation, hydroxylation/dehydroxylation, and disulfide bond formation and breakage.

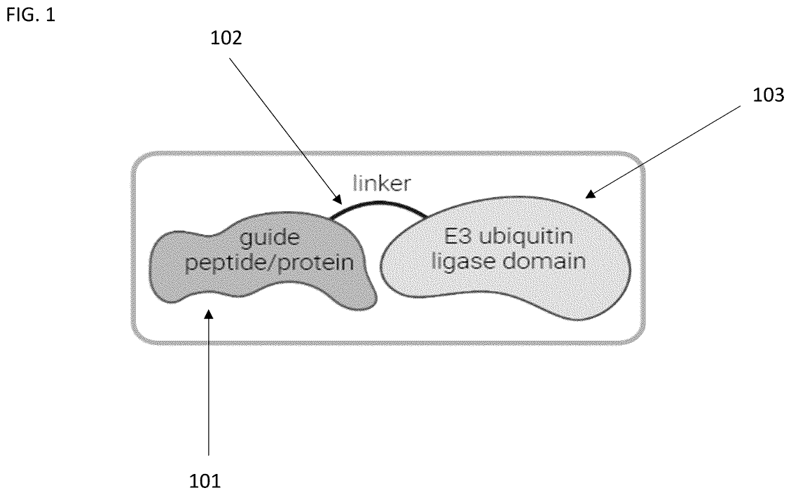

With more specific reference to FIG. 1, one or more particular implementations is directed to an isolated chimeric molecule comprising: a protein modulating domain 103 (e.g. kinase, phosphatase, acetylase, carboxylase, deubiquinase, glycosyltransferase); a linker; and (101) a targeting domain in which said targeting domain is capable of specifically directing the protein modulating domain to the target protein substrate where the targeting domain is heterologous to the protein modulating domain.

As shown in the particular illustration of FIG. 1, the protein modulating domain or PTMs 103 is a E3 ubiquitin ligase domain. However, in alternative configurations, any protein modulating domain can be used.

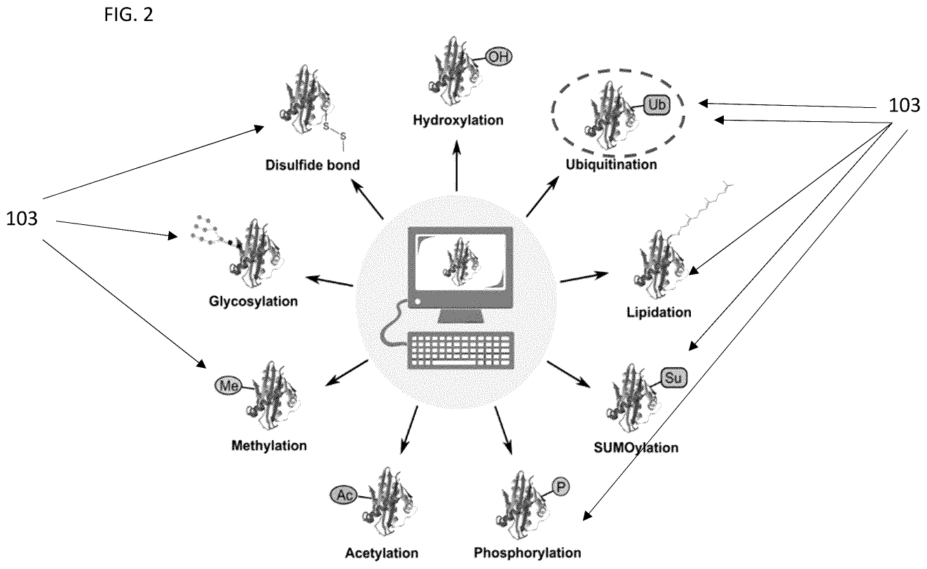

For example, as shown in FIG. 2, other PTMs processes such as acetylation, phosphorylation, SUMOylation, Lipidation, Hydroxylation, Glycosylation, Methylation, disulfide bonding, or ubiquitination are understood and appreciated.

The targeting domain 101, can, in one or more particular configurations, include an experimentally derived binder including, but not limited to affibodies, affilins, anticalins, atrimers, avimers, bicyclic peptides, cys-knots, DARPins, FN3, fynomers, kunitz domains, QBodies, monobodies, SCFV, VHH nanobodies, vNAR and VLR, or an algorithmically derived binder.

For example, one or more targeting domains can be derived using one or more computer implemented or directed processes that use or incorporate machine learning models. For example, U.S. Provisional Application No. 63/423,320, entitled: “Sequence-Based Framework to Design Peptide-Guided Degraders.”; U.S. Provisional Application No. 63/344,820, entitled: “Contrastive Learning for Peptide Based Degrader Design and Uses Thereof.”; U.S. Provisional Application No. 63/032,513, entitled: “Minimal Peptide Fusions for Targeted Intracellular Degradation.” and U.S. Patent Application US20140112922A1, entitled: “Targeted protein silencing using chimeras between antibodies and ubiquitination enzymes”, each describe the use of computational frameworks for generating a plurality of potential amino acid sequences that can link or bind to a given biological target, such as but not limited to Beta-catenin. Such amino sequences can be further fused to a PTMs, such as but not limited to E3 ubiquitin ligase. For example, one or more algorithmically generated sequences can be generated that bind the target protein and induce a post-translation modification when fused to a corresponding PTMs domain. In one or more implementations the computationally derived targeting domains are between 10-20 amino acids in length.

For example, one or more experimentally derived binders (101) can be obtained using a computational platform that identifies, optimizes and creates novel “guide peptide” binders to enable engineered proteins for use both inside and outside the cell, including for tasks such as protein modulating mRNA-based medicines. Such approaches have improved target specificity (mutant/isoform/PTM) relative to the existing art.

In a further example, the present disclosure also provides a chimeric molecule in which the targeting domain is computationally designed.

In one or more particular implementations, the computationally derived chimeric molecules described herein are directed to targeted protein stabilization (TPS). It will be appreciated by one of ordinary skill in the relevant art that TPS functions as an additional or alternative PTM process to targeted protein degradation. For example, as described herein, computationally derived chimeric molecules have been developed to engage in selective degradation of particular biological targets. However, the same biological target can be stabilized by changing the effector protein (103) portion of the computationally designed chimeric molecule.

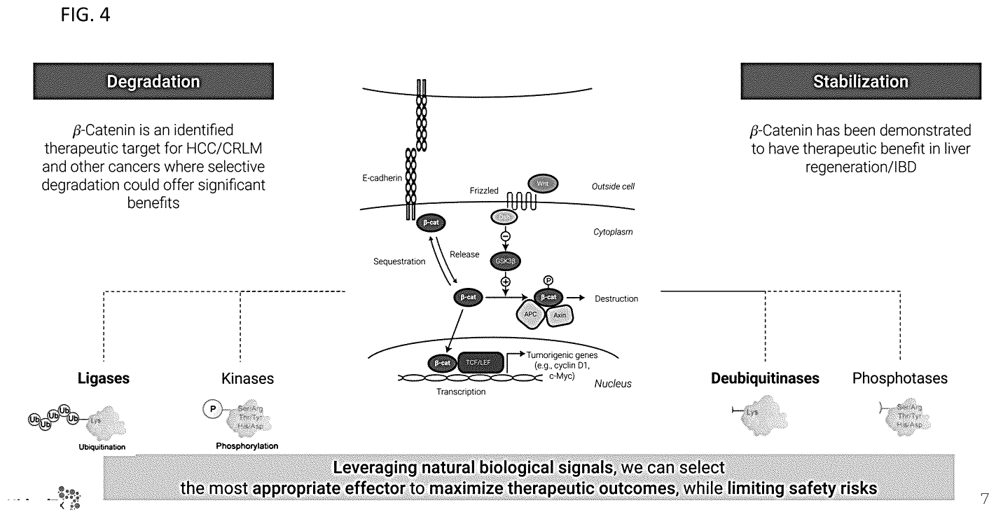

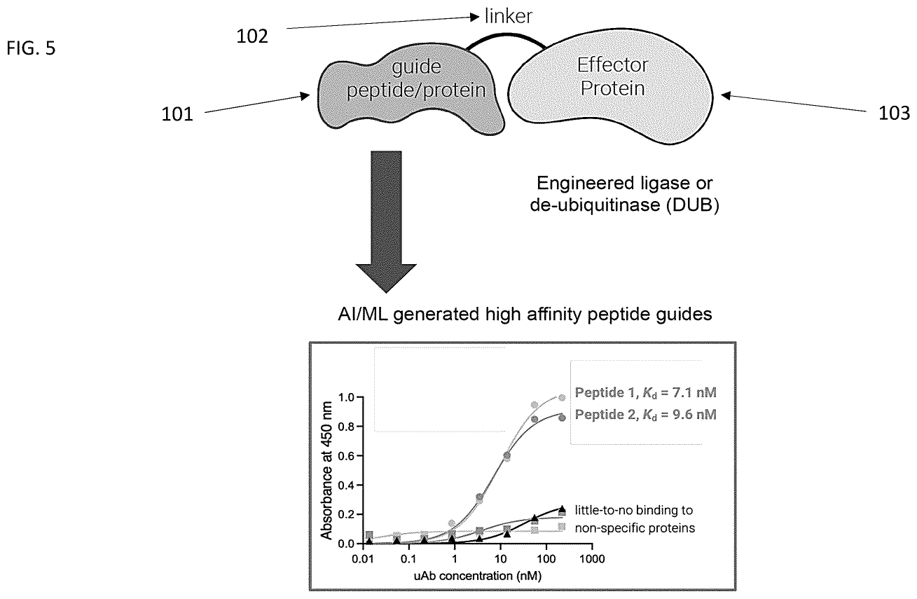

As shown in FIGS. 4-5, a biological target can be targeted for selected modification to maximize therapeutic outcomes. For instance, beta catenin can be modified by the computationally derived chimeric molecules described. In one instance, beta-catenin is targeted for selective degradation through ubiquitination or phosphorylation processes. Alternatively, the same target, here beta catenin, can be modified for stability, such as through the use of effector proteins, such as deubiquitinases or phosphatases enzymes. In one arrangement, a target-specific short “guide” peptides (101), designed using one or more sequence based algorithms, is fused to an effector molecule (103) (such as through a linker 102), such as a deubiquitinase, to yield a targeted protein stabilization chimeric molecule.

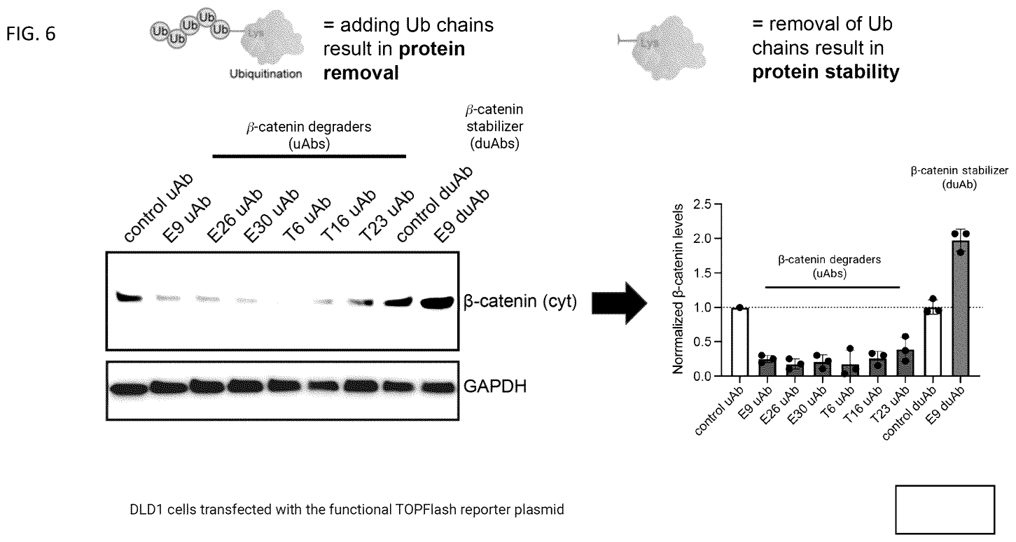

In one particular example, as shown in FIG. 6 the computationally derived chimeric molecule can be used to either stabilize or degrade a biological target of interest. For example, a TPS chimeric molecule is, in one arrangement, designed to stabilize Beta-catenin (β-catenin). For example, using the guide and linker sequences derived from protein language models, a sequence can be generated that causes exogenous and endogenous protein stabilization in a DUB-dependent manner.

For example, new target-binding peptides are readily designed via generative language models, SaLT&PepPr and PepPrCLIP described in, for example, U.S. Provisional Application No. 63/423,320, entitled: “Sequence-Based Framework to Design Peptide-Guided Degraders.”; U.S. Provisional Application No. 63/344,820, entitled: “Contrastive Learning for Peptide Based Degrader Design and Uses Thereof”, U.S. Provisional Application No. 63/032,513, and any PCT and/or national stage conversion applications which claim priority thereto, which are herein understood and incorporated by reference in their respective entireties.

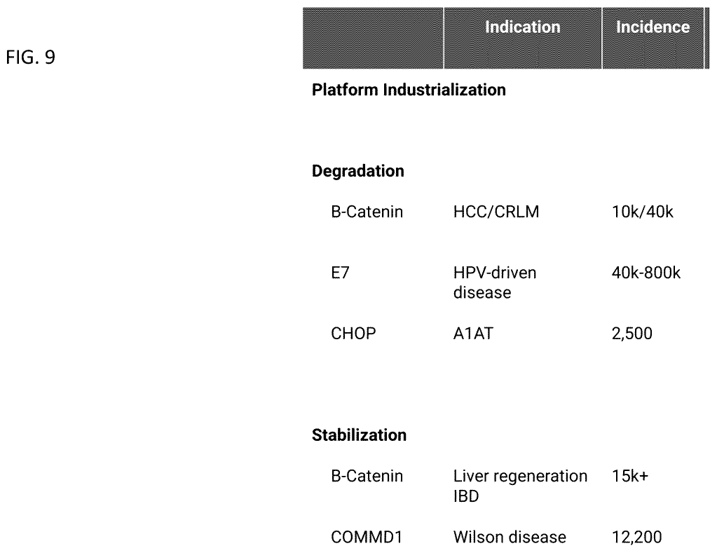

While the present example shown in FIG. 6 is specific to Beta-catenin (β-catenin), the computationally derived approach of generating a guide and linker sequences and fusing to an effector protein (such as but not limited to any of the PTMs described herein) will be understood and appreciated. For example, it will be appreciated and understood that other biological targets can be addressed for modification using the approaches and constructs described herein. For example, while the foregoing examples use β-catenin, the same chimeric PTM molecules can be used to degrade E7. CHOP or other therapeutic targets using similar chimeric construct designs as those developed for β-catenin. Likewise, while the present examples show stabilization of β-catenin using a guide peptide linked to deubiquitinases enzymes, the same constructs, suitably targeted, can be used to address other targets, such as COMMD1, as shown in FIG. 9.

By way of non-limiting example, an isolated chimeric molecule is provided where the chimeric molecules comprises: (i) a PTMs domain comprising a deubiquitinases motif without lysine residues; (ii) a targeting domain comprising a Beta-Catenin binding motif which is heterologous to the deubiquitinases motif; and (iii) a linker coupling said PTMs domain to said targeting domain.

By way of a further non-limiting example, an isolated chimeric molecule is provided where the chimeric molecules comprises: (i) a PTMs domain comprising a ubiquitinases motif without lysine residues; (ii) a targeting domain comprising a E7 binding motif which is heterologous to the ubiquitinases motif; and (iii) a linker coupling said PTMs domain to said targeting domain. In a further arrangement, the ubiquitinases motif comprises a eukaryotic U-box motif of an E3 ligase, wherein the U-box motif is a carboxyl terminus of human Hsc70-Interacting Protein (“CHIP (STUB1)”). In a further implementation, the degradation domain comprises Shigella flexneri E3 ligase, SspH1, SspH2, SlrP, AvrPtoB, LubX, NleG5-1, NleG2-3, LegU1, LegAU13, NIeL, SopA, SidC, XopL, GobX, VirF, GALA, AnkB, or SidE.

As shown in FIG. 5-6, both PTM and PTS can be accomplished using similar programmable architectures. For example, targeted-catenin degraders developed using the computationally designed architecture are shown to have improved degradation of β-catenin relative to control degraders. (See FIG. 6). Furthermore, using the same core architecture but replacing a ubiquitination effector protein with a deubiquitinating effector protein, the computationally designed chimeric molecule can be used to stabilize the biological target. For example, as shown in FIG. 6, when the chimeric molecule is designed to bind to beta-catenin and is linked to a deubiquitinating effector protein, the resulting chimeric molecule results in higher β-catenin levels relative to the control. (as shown further in FIG. 6). Thus, the described platform does not require undue experimentation to implement effective PTM or PTS of an identified target. That is, the chimeric construct (a computationally derived guide peptide/protein combined with a linker and an effector protein) can be designed to carry out multiple, and different, PTM effects with reasonable expectations of success.

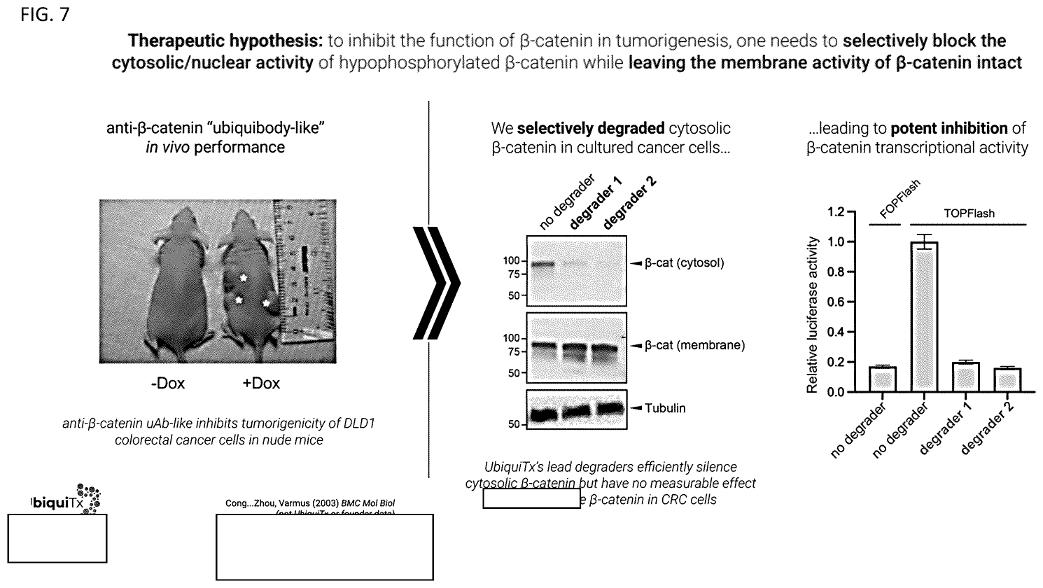

More particularly, as shown in FIG. 7, it will be understood that the chimeric construct developed herein can be used for selective targeting of a target of interest. For example, to inhibit the function of beta catenin in tumorigenesis it is determined by the inventors that the cytosolic/nuclear activity of hypophosphorylated beta catenin needs to be selectively blocked while leaving the membrane activity of beta catenin substantially unaffected.

For example, in one arrangement, the chimeric molecule is configured to treat a carcinoma where multiple mutation make the target extremely challenging for other therapies. For example, approaches that are directed to silencing of WT allele will deplete all β-catenin from cell, including critical membrane bound β-catenin, an undesired outcome. The presently described PTMs chimeric molecules are, in one or more configuration, adaptable to deplete the cytosolic β-catenin pool only with an E-cadherin mimic, thus showing that 100% degradation of all β-catenin is not required for efficacy in vivo.

In one or more implementations, a method is provided to inhibit the function of β-catenin in tumorigenesis. In one particular implementation, the method includes selectively blocking the cytosolic/nuclear activity of hypophosphorylated β-catenin while leaving the membrane activity of β-catenin intact. The method further includes administering to a patient in need thereof, an amount of peptide-E3 ligase fusion sufficient to selectively block the cytosolic/nuclear activity of hypophosphorylated β-catenin while leaving the membrane activity of β-catenin intact.

As described, it will also be appreciated that β-catenin stabilization can be achieved through the use of a chimeric molecule that has a PTMs domain comprising deubiquitinases motif(s). Here, such a chimeric molecule can be administered as part of a treatment regimen to patients suffering from severe alcoholic hepatitis (SAH). The inventors have found that hepatocyte proliferation and Wnt signaling is correlated with improved survival. For example, upregulation of Wnt signaling was implicated in improved liver function. Thus, in one treatment method, a chimeric molecule is administered to a patient suffering from SAH, wherein the chimeric molecule is specifically targeted to cytosolic β-catenin and includes a deubiquitinases motif.

In one or more implementations, the PTMs domain is selected from ubiquitin-specific proteases (USPs), ubiquitin C-terminal hydrolases (UCHs), ovarian tumor proteases (OTUs), Machado-Josephin domain proteases (MJDs), zinc-dependent metalloenzymes (JAMMs) and SUMO proteases. In one or more particular implementations, the PTMs domain includes one or more of ubiquitin C-terminal hydrolases USP13, USP14, USP38, USP39 or OTU proteases OTUB1 and OTUD6B.

In one or more implementations, the chimeric molecules incorporate one or more degrons (or short degradation inducing sequences) to manage the interaction of ubiquitin. For example, a chimeric molecule comprising a degron configured to interact or recruit E3 ligases and a targeting domain and a linker, linking the degron and the targeting domain is provided. Such a chimeric molecule can have an advantage of smaller genetic footprint. See Kim M S, Bhargava H K, Shavey G E, Lim W A, El-Samad H, Ng A H. Degron-based bioPROTACs for controlling signaling in CAR T cells. bioRxiv [Preprint]. 2024 Feb. 17:2024.02.16.580396. doi: 10.1101/2024.02.16.580396. PMID: 38405763; PMCID: PMC10888892, herein incorporated by reference as if presented in its respective entirety.

In yet a further implementation, the chimeric molecule is configured to degrade or aid in the degradation of chimeric antigen receptors (CAR) T cells. For instance, the described chimeric molecules can, in one arrangement, be configured to control or enhance CAR T cell signaling.

As shown in FIG. 7, the described computationally derived chimeric constructs are able to accomplish the selective degradation of cytosolic beta catenin but have no measurable effect on membrane beta catenin in CRC cells. Thus, the chimeric construct developed is able to selectively degrade cytosolic beta catenin in cultured cancer cells leading to potent inhibition of beta catenin transcriptional activity. Such an approach presents potential pathways for therapeutics for the treatment of forms and types of cancers. In one or more implementations a chimeric molecule configured with a PTMs domain, a targeting domain and a linker is administered to treat carcinoma. For example, the chimeric molecule described is configured to treat hepatocellular carcinoma, as shown in FIG. 9. In a further arrangement, the chimeric molecule described is designed to degrade cytosolic and membrane protein targets using either a nanobody or a synthetic construct, such as a leucine zipper.

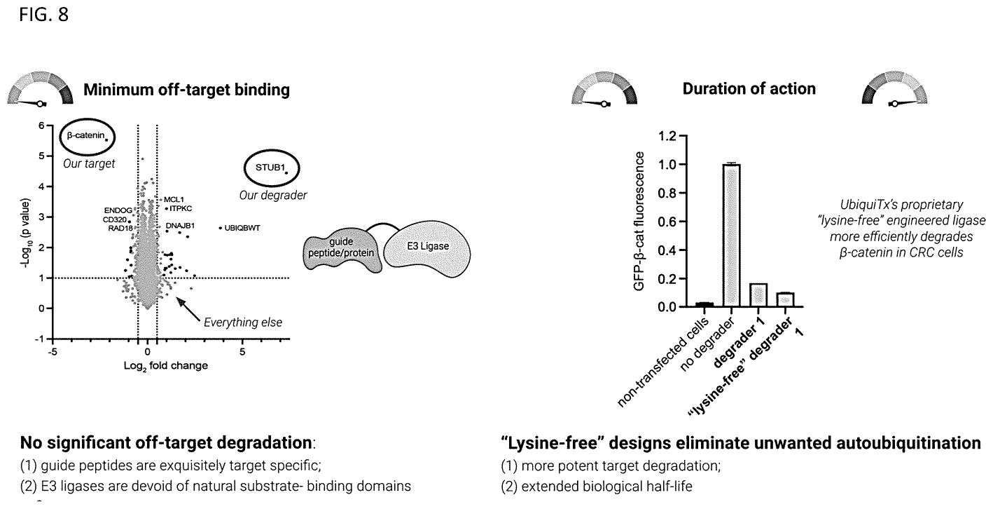

As further shown in FIG. 8, the designed targeted β-catenin degrader has insignificant off-target degradation. Thus, providing for a therapeutic having improved potential safety and efficacy.

It will be further appreciated that the chimeric molecules described herein can be integrated within targeted lipid nanoparticle platforms. Such approaches allow the chimeric molecules described hereinto to be encapsulated as mRNA and delivered to specific tissues of interest, with lower risk of potential side effects and toxicity.

In yet a further example, the present disclosure further provides a chimeric molecule in which the targeting domain is computationally designed and is relatively non-homologous to wild type binders to said target (e.g. a non-natural sequence).

In yet an alternative arrangement, the present disclosure also provides a chimeric molecule in which the PTM domain is computationally designed (e.g. a computationally designed enzyme). REF: Ych, A. H W., Norn, C., Kipnis, Y. et al. De novo design of luciferases using deep learning. Nature 614, 774-780 (2023). https://doi.org/10.1038/s41586-023-05696-3.

The isolated chimeric molecule can be targeted at intracellular or extracellular targets. Also disclosed are compositions as well as methods for treating a disease, substrate silencing, screening agents for therapeutic efficacy against a disease, and methods of screening for disease biomarkers. In one further example, can be directed to the degradation of cullin ring ligases.

In one or more particular implementations, a chimeric molecule is provide comprising: (i) at least one post translation modification (PTMs) domain; (ii) a targeting domain comprising a substrate-binding motif which is heterologous to the at least one PTMs domain; and (iii) a linker coupling said at least one PTMs domain to said targeting domain. The chimeric molecule of any of the previous implementations, wherein said linker is heterologous to the at least one PTMs domain and the targeting domain.

The chimeric molecule of claim 1 or claim 2, wherein said substrate-binding motif and/or said linker has no lysine residues.

The chimeric molecule of any of the previous implementations, wherein the at least one PTMs domain includes at least one enzyme directed to ubiquitination, deubiquination, lipidation, delipidation, SUMOylation, deSUMOylation, nitrosylation, denitrosylation, phosphporylation, dephosphorylation, acetylation, deacetylation, alkylation, dealklyation, methylation, demethylation, carboxylaton, decarboxylation, glycoysylation, deglycoylation, hydroxylation, dihydroxylation and disulfide bond formation and breakage.

The chimeric molecule of any of the previous implementations, wherein said PTMs domain is a degradation domain selected from one of an E3 ligase region, that includes E3A, mdm2, UBR5 (EDD1), CHIP (STUB1), LNXp80, CBX4, HACE1, HECTD1, HECTD2, HECTD3, HECW1, HECW2, HERC1, HERC2, HERC3, HERC4, HUWE1, ITCH, NEDD4, NEDD4L, PPIL2, PRPF19, PIAS1, PIAS2, PIAS3, PIAS4, RANBP2, RBX1, SMURF1, SMURF2, STUB, TOPORS, TRIP12, UBE3A, UBE3B, UBE3C, UBE4A (UFD2b), UBE4B (UFD2a), UBOX5 (UIP5), UBR5, WWP1, WWP2, ACT1 (TRAF3IP2), PUB19, PRP19 (PRPF19, SNEV), CYC4 (PPIL2, Cyp-60), and WDSUB1. In some embodiments, the U-box motif is selected from E3A, UBR5 (EDD1), CHIP (STUB1), STUB, UBE3A, UBE3B, UBE3C, UBE4A (UFD2b), UBE4B (UFD2a), UBOX5 (UIP5), UBR5, ACT1 (TRAF3IP2), PUB19, PRP19 (PRPF19, SNEV), CYC4 (PPIL2, Cyp-60), and WDSUB1.

The chimeric molecule of any of the previous implementations, wherein said targeting domain is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, V_{H}, V_{L}, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, VHH, knottin, DARPin, or Sso7d.

The chimeric molecule of any of the previous implementations, wherein said targeting domain is a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), EI3.4.3 (EGFR), E14.2.1 (EGFR), EI4.4.2 (EGFR), EI6.2.6 (EGFR), EI6.2.10 (EGFR), E246 (EGFR), C743 (CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), ml2.2.1 (mIgG), HA4 (AblSH2), HA10 (AblSH2), HA16 (AbISH2), HA18 (AblSH2), 159 (vEGFR), MUC16 (MSLN), E2 #3 (ERα/EF), E2 #4 (ERα/EF), E2 #5 (ERα/EF), E2 #6 (ERα/EF), E2 #7 (ERα/EF), E2 #8 (ERα/EF), E2 #9 (ERα/EF), E2 #10 (ERα/EF), E2 #11 (ERα/EF), E2 #23 (ERα/EF), E3 #2 (ERα/EF), E3 #6 (ERα/EF), OHT #31 (ERα/EF), OHT #32 (ERα/EF), OHT #33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozymc), BF4.9 (Lysozyme), BF4.4 (Lysozyme), BFslc4.01 (Lysozyme), BFslc4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), g12.5.3T88I (goat IgG), g12.5.2 (goat IgG), g12.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

The chimeric molecule of any of the previous implementations, wherein said substrate is an intracellular protein substrate.

The chimeric molecule of any of the previous implementations, wherein said substrate is selected from the group consisting of β-galactosidase, fluorescent protein, histone protein, nuclear localization signal (NLS), H-Ras protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

The chimeric molecule of any of the previous implementations, wherein the substrate is a fluorescent protein selected from the group consisting of green fluorescent protein, emerald fluorescent protein, venus fluorescent protein, cerulean fluorescent protein, and enhanced cyan fluorescent protein.

The chimeric molecule of any of the previous implementations, wherein said linker is a polypeptide linker of sufficient length to prevent the steric disruption of binding between said targeting domain and said protein substrate.

The chimeric molecule of any of the previous implementations, wherein said linker is not cleavable.

The chimeric molecule of any of the previous implementations, wherein said linker is enzymatically or hydrolytically cleavable.

A composition comprising: the chimeric molecule of claim 1; and a pharmaceutically-acceptable carrier.

The chimeric molecule of any of the previous implementations, including a second agent selected from the group consisting of an anti-inflammatory agent, an antidiabetic agent, a hypolipidemic agent, a chemotherapeutic agent, an antiviral agent, an antibiotic, a metabolic agent, a small molecule inhibitor, a protein kinase inhibitor, adjuvants, apoptotic agents, a proliferative agent, and organotropic targeting agents, and any combination thereof.

A method of treating a disease comprising: administering the composition of any of the previous implementations, to a subject having a disease.

A method of the previous implementations, wherein the subject to whom said composition is administered has an increased expression level of said protein substrate compared to a subject not afflicted with said disease.

A method of the previous implementations, wherein said disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

The method any of the previous implementations, wherein the administering is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

The present disclosure also includes a chimeric molecule in which the targeting domain is computationally designed. Furthermore, the chimeric molecule of any of the previous implementations is provided in which the PTMs domain and the targeting domain are computationally designed and both the PTMs domain and the targeting domain are relatively non-homologous to wild type binders to said target.

It will be appreciated that the chimeric molecules described herein are an effective degrader in various models, such as mECSs.

The described approaches, concepts and chimeric molecules can be used in connection with engineered T cells, as well as other cell types, including human stem cells, natural killer (NK) cells and macrophages.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

Any patents, patent applications, publications or other references are herein incorporated by reference as if each was presented in its respective entirety.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments, but should be defined only in accordance with the following claims and their equivalents.

What is claimed is:

1. A chimeric molecule comprising:
(i) at least one post translation modification (PTMs) domain;
(ii) a targeting domain comprising a substrate-binding motif which is heterologous to the at least one PTMs domain; and
(iii) a linker coupling said at least one PTMs domain to said targeting domain.

2. The chimeric molecule of claim 1, wherein said linker is heterologous to the at least one PTMs domain and the targeting domain.

3. The chimeric molecule of claim 1, wherein said substrate-binding motif and/or said linker has no lysine residues.

4. The chimeric molecule of claim 1, wherein the at least one PTMs domain includes at least one enzyme directed to ubiquitination, deubiquination, lipidation, delipidation, SUMOylation, deSUMOylation, nitrosylation, denitrosylation, phosphporylation, dephosphorylation, acetylation, deacetylation, alkylation, dealklyation, methylation, demethylation, carboxylaton, decarboxylation, glycoysylation, deglycoylation, hydroxylation, dihydroxylation and disulfide bond formation and breakage.

5. The chimeric molecule of claim 1, wherein said PTMs domain is a degradation domain selected from one of an E3 ligase region, that includes E3A, mdm2, UBR5 (EDD1), CHIP (STUB1), LNXp80, CBX4, HACE1, HECTD1, HECTD2, HECTD3, HECW1, HECW2, HERC1, HERC2, HERC3, HERC4, HUWE1, ITCH, NEDD4, NEDD4L, PPIL2, PRPF19, PIAS1, PIAS2, PIAS3, PIAS4, RANBP2, RBX1, SMURF1, SMURF2, STUB, TOPORS, TRIP12, UBE3A, UBE3B, UBE3C, UBE4A (UFD2b), UBE4B (UFD2a), UBOX5 (UIP5), UBR5, WWP1, WWP2, ACT1 (TRAF3IP2), PUB19, PRP19 (PRPF19, SNEV), CYC4 (PPIL2, Cyp-60), and WDSUB1. In some embodiments, the U-box motif is selected from E3A, UBR5 (EDD1), CHIP (STUB1), STUB, UBE3A, UBE3B, UBE3C, UBE4A (UFD2b), UBE4B (UFD2a), UBOX5 (UIP5), UBR5, ACT1 (TRAF3IP2), PUB19, PRP19 (PRPF19, SNEV), CYC4 (PPIL2, Cyp-60), and WDSUB1.

6. The chimeric molecule of claim 1, wherein said targeting domain is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, VHH, knottin, DARPin, or Sso7d.

7. The chimeric molecule of claim 6, wherein said targeting domain is a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), EI3.4.3 (EGFR), EI4.2.1 (EGFR), EI4.4.2 (EGFR), EI6.2.6 (EGFR), EI6.2.10 (EGFR), E246 (EGFR), C743 (CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), ml2.2.1 (mIgG), HA4 (AblSH2), HA10 (AblSH2), HA16 (AblSH2), HA18 (AblSH2), 159 (vEGFR), MUC16 (MSLN), E2 #3 (ERα/EF), E2 #4 (ERα/EF), E2 #5 (ERα/EF), E2 #6 (ERα/EF), E2 #7 (ERα/EF), E2 #8 (ERα/EF), E2 #9 (ERα/EF), E2 #10 (ERα/EF), E2 #11 (ERα/EF), E2 #23 (ERα/EF), E3 #2 (ERα/EF), E3 #6 (ERα/EF), OHT #31 (ERα/EF), OHT #32 (ERα/EF), OHT #33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFslc4.01 (Lysozyme), BFslc4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), g12.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

8. The chimeric molecule of claim 1, wherein said substrate is an intracellular protein substrate.

9. The chimeric molecule of claim 8, wherein said substrate is selected from the group consisting of β-galactosidase, fluorescent protein, histone protein, nuclear localization signal (NLS), H-Ras protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

10. The chimeric molecule of claim 9, wherein the substrate is a fluorescent protein selected from the group consisting of green fluorescent protein, emerald fluorescent protein, venus fluorescent protein, cerulean fluorescent protein, and enhanced cyan fluorescent protein.

11. The chimeric molecule of claim 10, wherein said linker is a polypeptide linker of sufficient length to prevent the steric disruption of binding between said targeting domain and said protein substrate.

12. The chimeric molecule of claim 11, wherein said linker is not cleavable.

13. The chimeric molecule of claim 12, wherein said linker is enzymatically or hydrolytically cleavable.

14. A composition comprising:
the chimeric molecule of claim 1; and
a pharmaceutically-acceptable carrier.

15. The composition of claim 1 further comprising:
a second agent selected from the group consisting of an anti-inflammatory agent, an antidiabetic agent, a hypolipidemic agent, a chemotherapeutic agent, an antiviral agent, an antibiotic, a metabolic agent, a small molecule inhibitor, a protein kinase inhibitor, adjuvants, apoptotic agents, a proliferative agent, and organotropic targeting agents, and any combination thereof.

16. A method of treating a disease comprising:
administering the composition of claim 15 to a subject having a disease, wherein the subject to whom said composition is administered has an increased expression level of said protein substrate compared to a subject not afflicted with said disease.

17. The method of claim 15, wherein said disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

18. The method of claim 16, wherein the administering is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

19. A chimeric molecule in which the targeting domain is computationally designed.

20. A chimeric molecule of claim 1, in which the PTMs domain and the targeting domain are computationally designed and both the PTMs domain and the targeting domain are relatively non-homologous to wild type binders to said target.
